(19) United States
(12) Patent Application Publication
(10) Pub. No.: US 2002/0198393 A1
(43) Pub. Date: Dec. 26, 2002

(54) Economic and other benefits of refining vegetable oil with potassium hydroxide

(76) Inventors: Ralph S. Daniels, Sherman, TX (US); Monoj K. Gupta, Richardson, TX (US); Phillip L. McKinney, Sherman, TX (US)

Correspondence Address: BLODGETT BLODGETT, 43 HIGHLAND STREET, WORCESTER, MA, 016092797

(21) Appl. No.: 09/992,553

(22) Filed: Nov. 6, 2001

Related U.S. Application Data
(63) Continuation of application No. PCT/US00/12314, filed on May 4, 2000.
(60) Provisional application No. 60/133,220, filed on May 7, 1999.

(51) Int. Cl. C11B 3/00; C07C 51/43

(52) U.S. Cl. 554/178

(57) ABSTRACT

In refining crude vegetable oils, there are certain economic and quality advantages to refining the oils with potassium hydroxide. Lower overall cost is achieved from reduced levels of soap and trace impurities in the oil, improved refining yield for the oils refined with potassium hydroxide, higher retention of recoverable tocopherols in the oil, gentler reaction with triglyceride, lower mono and diglyceride in the potassium hydroxide soapstock, the option to convert a by-product of soapstock acidulation, into a profitable co-product and eliminate acid-water discharge from the soapstock acidulation process.

### INTRODUCTION

In chemical refining process sodium hydroxide is used for neutralizing vegetable oils. Although other alkali's, such as soda ash, potassium hydroxide, caesium hydroxide have been tried in the past, none of them developed into standard industrial practice. Soda ash and calcium hydroxide did not produce the desired results. Potassium hydroxide has been avoided by the oil industry because of its high cost.

Although sodium hydroxide treatment is viewed by the oil industry as the economic means to refine crude vegetable oils, our studies are indicating certain economic as well as quality advantages of refining vegetable oils with potassium hydroxide. Plant trials as well as laboratory experiments have been conducted on cottonseed and soybean oils to investigate the benefits of potassium hydroxide refining. The results have been positive in both instances. Refining with potassium hydroxide has exhibited potential economic advantages that can significantly reduce the cost of oil processing.

Lower overall cost of oil processing is achieved from reduced levels of soap and trace impurities in the oil and improved refining yield for the oils refined with potassium hydroxide.

In addition, higher retention of tocopherols in the oil, refined with potassium hydroxide, could lead into a higher tocopherol recovery from the distillate. This can generate substantially higher revenue.

Potassium hydroxide is found to be gentler with triglyceride, the main constituent of the oil.

The test revealed that potassium hydroxide was less reactive than sodium hydroxide in reacting with the neutral oil in the soapstock. This could mean that potassium hydroxide can improve refining yield.

Besides the presence of higher level of monoglyceride and diglyceride in the oil could lead into higher oil loss due to hydrolysis, emulsification and oxidative polymerization during process.

Higher monoglyceride and diglyceride in the oil can significantly lower its fry life.

Higher mono and diglyceride in the soapstock in sodium hydroxide refining might increase the tendency to form the middle phase and hinder separation of acid-oil from acid-water.

Lower mono and diglyceride in the potassium hydroxide soapstock may also imply that this alkali would reduce oil loss when poor quality crude requires excess caustic treat in the refining process.

Conversely, lower mono and diglyceride in potassium soapstock could mean that acidulation and separation of the acid-oil and acid-water could be easier,

Soapstock from the potassium hydroxide process can be acidulated with sulfuric acid, as normal. Acid water can be neutralized with ammonia or ammonium hydroxide and the resultant material can be used as a nutrient to plants because the product is rich in potassium, nitrogen, phosphorus and sulfur, and other minor nutrients. This allows the oil processor to convert a by-product of soapstock acidulation, into a profitable co-product and eliminate acid-water discharge from the soapstock acidulation process.

TABLE I
Laboratory Refining Conditions on Crude Soybean Oil (Test #1)
(Work Conducted at Texas A&M Laboratory)

| DESCRIPTION | NaOH REFINING | KOH REFINING |
|---|---|---|
| Batch Size | 1000 grams | 1000 grams |
| Refining | | |
| Alkali Strength, ° Be | 12 | 11.3 |
| % Concentration | 8.08 | 9.5 |
| % Excess | 0.2 | 0.2 |
| Temperature, ° F. | 160 | 160 |
| Time of Mixing | 15 | 15 |
| Centrifuging | | |
| Type/Model | Sorvall Superspeed | Sorvall Superspeed |
| | Model RC2-B with Temp. | Model RC2-B with Temp. |
| | Control | Control |
| RPM | 10,000 | 10,000 |
| Time, min. | 30 | 30 |
| Water Washing | | |
| Water/Oil Mix temp. | 171° F. | 171° F. |
| Centrifuge RPM | 10,000 | 10,000 |
| Time, min. | 30 | 30 |

TABLE II
Observations during Refining (Test #1)

| Observations | NaOH Refining | KOH Refining |
|---|---|---|
| Visibility of soap in oil | Not very visible | Distinctly visible |
| Distribution in oil | Most of the soapstock had | Soapstock was distributed |
| | accumulated art the bottom | throughout the body of the |
| | of the Refining Kettle | oil. In addition, some were |
| | | floating and some were |
| | | found at the bottom of the |
| | | Refining Kettle |
| Appearance | Very viscous mass. | Very rigid or elastic in |
| | Hard to work with. | appearance |
| | A lot of oil was occluded in | Easy to handle |
| | the soapstock | Not much occluded oil was |
| | | visible in the soapstock |

TABLE III
Laboratory Refining Data on Crude Soybean Oil (Test #1)
(Work Conducted at Texas A&M Laboratory)

| | | | ABSOLUTE | |
|---|---|---|---|---|
| | | | DIFFERENCE | PER- |
| | | | (FOR KOH | CENT |
| | NaOH | KOH | REFINED | DIFFER- |
| ANALYSIS | REFINING | REFINING | OILS) | ENCE |
| % FFA in | 0.496 | 0.496 | | |
| Crude | | | | |
| Before Citric | | | | |
| Acid Treatment | | | | |
| After Citric | 0.58 | 0.62 | | |
| Acid Treatment | | | | |
| % FFA, in | 0.05 | 0.04 | −0.01 | −20% |
| Refined Oil | | | | |
| Water Washed | 0.035 | 0.03 | −0.005 | −14% |
| Oil | | | | |
| PPM Soap, in | 240 | 115 | −125 | −52% |
| Refined Oil | | | | |
| Water Washed | 27.4 | 12.2 | −15.2 | −55% |
| Oil | | | | |
| Refining loss, | 6 | 5 | −1 | −16.7% |
| % | | | | |
| Batch Basis | | | | |
| Apparent | 6352 | 5749 | −603 | −9.4% |
| Viscosity Of | | | | |
| Soapstock* | | | | |
| At 140° F. in | | | | |
| CP | | | | |
| @ | | | | |
| 0.4 RPM | | | | |

*Brookfield Viscometer, Model: RV, Spindle: CP 40: Temperature 140° F.

TABLE IV
Trace component Analyses In Laboratory Refined Crude Soybean Oil
(Test #1) (Work Conducted at Texas A&M Laboratory)

| | | | | DIFFERENCE |
|---|---|---|---|---|
| | | | | (FOR KOH |
| | CRUDE | NaOH | KOH | REFINED |
| ANALYSIS | OIL | REFINING | REFINING | OIL) |
| Tocopherols, | 1313 | 1257 | 1285 | +28 ppm |
| ppm | | | | |
| Iron, ppm | 0.4 | <0.4 | <0.4 | NONE |
| Phosphorus, | 460 | | | |
| ppm | | | | |
| Calcium, ppm | 46 | 15 | 12 | ,−3 |
| Magnesium, | 25 | 6 | 4 | −2 |
| ppm | | | | |
| Monoglyceride, | 0.58 | 0.44 | 0.33 | −0.11 |
| % | | | | |
| Diglyceride, % | 3.9 | 3.17 | 2.42 | −0.75 |

TABLE V
Impact of Storage Time on Soapstock Viscosity (Test #1)
(Work Conducted at Texas A&M Laboratory)

| DESCRIP- | NaOH | KOH | ABSOLUTE | PERCENT |
|---|---|---|---|---|
| TION | REFINING | REFINING | DIFFERENCE | DIFFERENCE |
| Viscosity in CP | 6352 | 5749 | −603 | −9.4% |
| Fresh | | | | |
| Soapstock | | | | |
| @ | | | | |
| 0.4 RPM | | | | |
| Viscosity in CP | 6048 | 5343 | −705 | −10.03% |
| 1-Day Old | | | | |
| Soapstock | | | | |
| @ | | | | |
| 0.4 RPM | | | | |
| Viscosity in CP | 6160 | 5365 | −794 | −12.7% |
| 7-Days Old | | | | |
| Soapstock | | | | |
| @ | | | | |
| 0.4 RPM | | | | |

TABLE VI
Effect of Storage Time on Neutral Oil in Soapstock (test #1)
(Work Conducted at Texas A&M Laboratory)

| | | After 2 Hours of | After 5 hours of |
|---|---|---|---|
| Analyses | Fresh Soapstock | Storage | Storage |
| Sodium Soap | | | |
| % Moisture | 38 | 38 | 37 |
| % Neutral Oil | | | |
| As is | − | 14.7 | 11.5 |
| Dry Basis | − | 21.9 | 18.1 |
| Potassium Soap | | | |
| % Moisture | 37 | 38 | 38 |
| % Neutral Oil | | | |
| As is | 23.1 | 24.2 | 24 |
| Dry Basis | 36.8 | 39.03 | 38.7 |

Note: The neutral oil content in the KOH soap did not change in 5 hours. This indicates KOH is gentler on neutral oil.

TABLE V
Mono & Diglyceride Content In The Neutral Oil Present In Soapstock

| | CRUDE OIL | NaOH REFINING | KOH REFINING |
|---|---|---|---|
| % Monoglyceride | 0.58 | 2.03 | 1.19 |
| % Diglyceride | 3.9 | 8.13 | 3.75 |

Note: The higher mono and diglyceride content of the neutral oil in the soapstock for NaOH refining implies stronger alkali activity for NaOH. One could infer that similar reactions occur when the crude oil is over-refined where the impact goes unnoticed.

TABLE VIII
Trace Component Analyses on Soybean Oil From Extended
Reaction Time (Test #2)
(Work Conducted at Texas A&M Laboratory)

| | | | | |
|---|---|---|---|---|
| Extended | % FFA in | % | | PPM, |
| Reaction With | Refined Oil | Monoglycerides | % Diglycerides | Tocopherols |

NaOH

| | | |
|---|---|---|
| 30 min | 0.015 | 1230 |
| 60 min | 0.025 | 1218 |
| 2 hrs | 0.035 | 1202 |
| 5 hrs | 0.065 | 1144 |
| KOH | | |
| 30 min | 0.03 | 1310 |
| 60 min | 0.05 | 1331 |
| 2 hrs | 0.05 | 1316 |
| 5 hrs | 0.07 | 1308 |

Note:
1. Difference in tocopherols between NaOH and KOH treatment is directionally higher for KOH treated oil. This needs additional data to confirm if this difference is significant.
2. Increase in % FFA in refined oil with longer reaction time could have been caused due to hydrolysis of the neutral oil in presence of the soap and water.

1. A process of refining agricultural oils on a commercial scale in which refined oil is separated from soapstock by addition of a caustic, characterized by the fact that: the caustic employed is predominantly potassium hydroxide.

2. A process as recited in claim 1, further characterized by the fact that: the caustic employed consists essentially of potassium hydroxide.

3. A process as recited in claim 1 or 2, further characterized by the fact that: the retention of recoverable tocopherols is increased.

4. A process as recited in claim 1 or 2, further characterized by the fact that: the level of mono and diglycerides in the soapstock is decreased.
